(19) United States
(12) Patent Application Publication
(10) Pub. No.: US 2011/0138544 A1
(43) Pub. Date: Jun. 16, 2011

(54) Dyeing composition comprising a particular hydrophobic dye and a particular propylene glycol derivatives

(76) Inventors: Maxime De Boni, Tokyo (JP); Alain Lagrange, Coupvray (FR)

(21) Appl. No.: 11/919,510

(22) PCT Filed: Apr. 27, 2006
(86) PCT No.: PCT/FR2006/000955

§ 371 Date: Sep. 4, 2009

Related U.S. Application Data
(60) Provisional application No. 60/677,348, filed on May 4, 2005.

(30) Foreign Application Priority Data
Apr. 27, 2005 (FR) 0551085

(51) Int. Cl. A61K 8/39; A61K 8/34; A61K 8/49; A61K 8/41; A61K 8/42; A61Q 5/10

(52) U.S. Cl. 8/407; 8/405

(57) ABSTRACT

The present invention relates to a dye composition comprising, in a suitable dyeing medium, at least one hydrophobic direct dye whose logP is greater than 2, the suitable medium containing water, and at least one propylene glycol derivative of formula (I) below

R₁(OC₃H₆)ₙOR₂

This composition makes it possible to obtain strong colorations. Furthermore, the coloration obtained makes it possible to achieve or even exceed the fastness obtained by oxidation dyeing.

The invention relates to a dye composition comprising, in a suitable medium, a particular hydrophobic dye and one or more particular propylene glycol derivatives. The invention also relates to the use of this composition for dyeing keratin fibres and also to the dyeing process using this composition.

It has been sought for a long time to modify the colour of hair and in particular to cover grey hair. Several techniques have been developed to do this.

It is known practice to dye keratin materials, and in particular human hair, with dye compositions containing direct dyes. The standard dyes that are used are in particular dyes of the nitrobenzene, anthraquinone, nitropyridine, azo, xanthene, acridine, azine or triarylmethane type or natural dyes. These dyes may be nonionic, anionic, cationic or amphoteric.

These dyes, which are coloured and colouring molecules that have affinity for keratin fibres, are applied for a time that is necessary to obtain the desired coloration, and are then rinsed out.

The colorations resulting therefrom are particularly chromatic, but are, however, temporary or semi-permanent since the nature of the interactions that bind the direct dyes to the keratin fibre and their desorption from the surface and/or the core of the fibre are responsible for their weak dyeing power and their poor wash-fastness or perspiration-fastness.

Moreover, it is known practice to dye keratin fibres permanently by oxidation dyeing. This dyeing technique consists in applying to the keratin fibres a composition containing dye precursors such as oxidation bases and couplers. Under the action of an oxidizing agent, these precursors form one or more coloured species in the hair.

The variety of molecules used as oxidation bases and couplers allows a wide range of colours to be obtained. The colorations resulting therefrom are permanent, strong and resistant to external agents, especially to light, bad weather, washing, perspiration and rubbing. However, this type of dyeing results in degradation of the fibre due to the use of an oxidizing agent.

There is still a need to develop novel direct dye compositions for obtaining varied shades, in particular pastels shades, and which show good fastness, especially with respect to external agents such as light, shampoo and sweat, while at the same time preserving the quality of the keratin fibres. In particular, there is a need to develop dye compositions that allow the production of colorations whose fastness is close to the coloration obtained by oxidation, without the drawbacks associated with the presence of an oxidizing agent.

This aim is achieved with the present invention, one subject of which is a cosmetic dye composition comprising, in a suitable cosmetic dyeing medium, at least one hydrophobic direct dye whose logP is greater than 2, the suitable medium containing at least 40% by weight of water relative to the total weight of the dye composition, and at least one propylene glycol derivative of formula (I) below

R₁(OC₃H₆)ₙOR₂

in which R₁ represents hydrogen or a C₁-C₆ alkyl or C₂-C₆ acyl radical, R₂ represents a C₁-C₆ alkyl or C₆-C₃₀ aryl radical and n ranges from 1 to 6 inclusive, the OC₃H₆ unit possibly being linear or branched.

The composition of the invention makes it possible to obtain varied shades and strong colorations. Furthermore, the coloration obtained makes it possible to achieve or even exceed the fastness obtained by oxidation dyeing. Thus, the coloration obtained is very resistant to external agents, especially to repeated washing.

A subject of the invention is also the use, for the dyeing of keratin fibres, of a composition comprising, in a suitable cosmetic dyeing medium, at least one hydrophobic direct dye whose logP is greater than 2, the suitable medium containing water, and at least one propylene glycol derivative of formula (I) below

R₁(OC₃H₆)ₙOR₂ (I)

in which R₁ represents hydrogen or a C₁-C₆ alkyl or C₂-C₆ acyl radical, R₂ represents a C₁-C₆ alkyl or C₆-C₃₀ aryl radical and n ranges from 1 to 6 inclusive, the OC₃H₆ unit possibly being linear or branched.

In the context of the invention, the logP value conventionally represents the partition coefficient of the dye between octanol and water. The logP value may be calculated according to the method described in the article by Meylan and Howard “Atom/Fragment contribution method for estimating octanol-water partition coefficient”, J. Pharm. Sci., 84: 83-92, 1995. This value may also be calculated using a range of software available on the market that determines the logP value as a function of the structure of a molecule. For example, mention may be made of the Epiwin software from the United States Environmental Agency.

The direct dyes that may be used in the composition of the invention are hydrophobic dyes known in the art, which have a logP value of greater than 2. Examples that may be mentioned include:

| Dye | Chemical structure | logP |
|---|---|---|
| Disperse Red 13 | 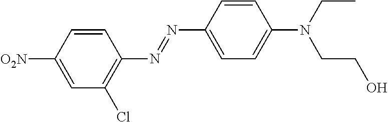 | 5.22 |
| Disperse Green 9 | 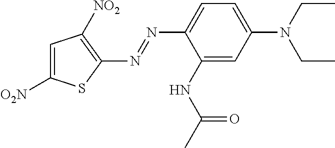 | 4.23 |
| Solvent Black 3 | 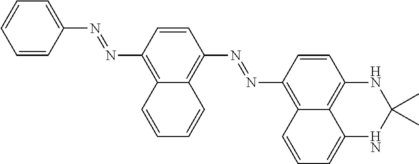 | 7.50 |
| Disperse Blue 148 | 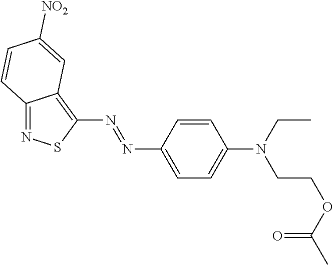 | 4.81 |
| Disperse Violet 63 | 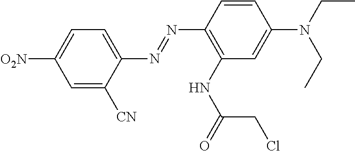 | 5.30 |
| Disperse Blue 60 | 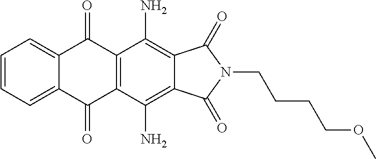 | 3.38 |
| Disperse Blue 14 | 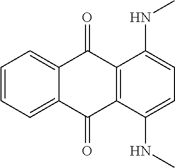 | 4.25 |
| Solvent Orange 15 | 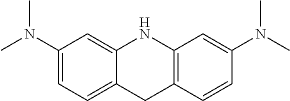 | 3.90 |
| Solvent Orange 7 | 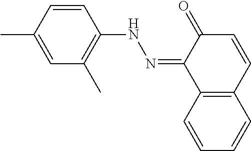 | 4.40 |
| Solvent Blue 14 | 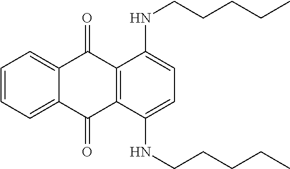 | 8.18 |
| Disperse Yellow 82 | 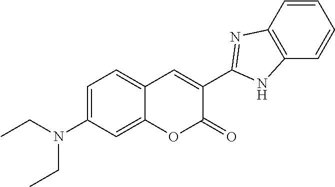 | 3.68 |

According to one particular embodiment, a logP of the dye that is useful in the composition of the invention is greater than 4.

The direct dye(s) with a logP of greater than 2 may be present in the composition in amounts of between 0.001% and 10% by weight approximately relative to the total weight of the composition.

In the composition of the invention, for the propylene glycol derivative of formula (I), the term “alkyl radical” means linear or branched radicals such as methyl, ethyl, propyl, isopropyl, isobutyl, tert-butyl, pentyl and hexyl radicals. Aryl radicals that may especially be mentioned include phenyl and benzyl radicals, etc.

In formula (I), the OC₃H₆ unit represents, for example, OCH2CH2CH2, OCH2CH(CH3) or OCH(CH3)CH2.

Propylene glycol derivatives of formula (I) that may be mentioned include the following propylene glycols;

| | |
|---|---|
| Dipropylene glycol methyl ether | CH₃O(C₃H₆O)₂H |
| (Dowanol DPM) | |
| Tripropylene glycol methyl ether | CH₃O(C₃H₆O)₃H |
| (Dowanol TPM) | |
| Propylene glycol methyl ether acetate | CH₃OC₃H₆OCOCH₃ |
| (Dowanol PMA) | |
| Dipropylene glycol methyl ether acetate | CH₃O(C₃H₆O)₂COCH₃ |
| (Dowanol DPMA) | |
| Propylene glycol n-propyl ether | C₃H₇OCH₂CH(CH₃)OH |
| (Dowanol PnP) | |
| Propylene glycol n-butyl ether | C₄H₉OCH₂CH(CH₃)OH |
| (Dowanol PnB) | |
| Propylene glycol phenyl ether | C₆H₅OC₃H₆OH |
| (Dowanol PPh) | |
| Dipropylene glycol n-propyl ether (DPnP) | C₃H₇O[CH₂(CH)CH₃O]₂H |
| Tripropylene glycol n-propyl ether (TPnP) | C₃H₇O[CH₂(CH)CH₃O]₃H |
| Dipropylene glycol n-butyl ether (DPnB) | C₄H₉O[CH₂(CH)CH₃O]₂H |
| Tripropylene glycol n-butyl ether (TPnB) | C₄H₉O[CH₂(CH)CH₃O]₃H |
| Dipropylene glycol dimethyl ether (DMM) | CH₃O(C₃H₆O)₂CH₃ |

According to one particular embodiment, the propylene glycol derivative of formula (I) is such that n ranges from 1 to 4 and R₁ represents a C₁-C₆ and preferably C₁-C₄ alkyl radical.

Preferably, if R₁ represents hydrogen, then n is greater than 1, and if n is equal to 1, then R₂ represents a C₂-C₆ alkyl radical.

The composition of the invention generally comprises an amount of propylene glycol derivatives of formula (I) of between 0.1% and 40%, preferably between 0.5% and 30% and even more preferentially from 1% to 20% by weight relative to the total weight of the composition.

According to one particular embodiment, the medium that is suitable for dyeing keratin fibres comprises at least 70% by weight of water relative to the total weight of the dye composition.

The dyeing medium may be constituted, for example, solely of water or of a mixture of water and of at least one organic solvent other than the propylene glycol derivative of formula (I). Examples of additional organic solvents that may be mentioned include C₁-C₄ lower alkanols, such as ethanol and isopropanol; polyols and polyol ethers, for instance 2-butoxyethanol and diethylene glycol monomethyl ether and monoethyl ether, and also aromatic alcohols, for instance benzyl alcohol or phenoxyethanol, and mixtures thereof.

For the dyeing of human keratin fibres, the dyeing medium is a suitable cosmetic medium.

The total amount of solvent, including the propylene glycol derivative(s) of formula (I), may range between 0.1% and 80% by weight approximately, preferably between 0.5% and 50% by weight approximately and more preferably between 1% and 30% by weight, relative to the total weight of the composition.

The dye composition in accordance with the invention may also contain direct dyes other than the direct dyes that are useful in the present invention. These additional direct dyes are, for example, neutral, acidic or cationic nitrobenzene direct dyes, neutral, acidic or cationic azo direct dyes, neutral, acidic or cationic quinone and in particular anthraquinone direct dyes, azine direct dyes, triarylmethane direct dyes, indoamine direct dyes and natural direct dyes.

Among the benzene-based direct dyes, mention may be made, in a non-limiting manner, of the following compounds:
1,4-diamino-2-nitrobenzene
1-amino-2-nitro-4-(β-hydroxyethylamino)benzene
1-amino-2-nitro-4-bis(β-hydroxyethyl)aminobenzene
1,4-bis(β-hydroxyethylamino)-2-nitrobenzene
1-β-hydroxyethylamino-2-nitro-4-bis(β-hydroxyethyl-amino)benzene
1-β-hydroxyethylamino-2-nitro-4-aminobenzene
1-β-hydroxyethylamino-2-nitro-4-(ethyl)(β-hydroxy-ethyl)aminobenzene
1-amino-3-methyl-4-β-hydroxyethylamino-6-nitrobenzene
1-amino-2-nitro-4-β-hydroxyethylamino-5-chlorobenzene
1,2-diamino-4-nitrobenzene
1-amino-2-β-hydroxyethylamino-5-nitrobenzene
1,2-bis(β-hydroxyethylamino)-4-nitrobenzene
1-amino-2-[tris(hydroxymethyl)methylamino]-5-nitro-benzene
1-hydroxy-2-amino-5-nitrobenzene
1-hydroxy-2-amino-4-nitrobenzene
1-hydroxy-3-nitro-4-aminobenzene
1-hydroxy-2-amino-4,6-dinitrobenzene
1-β-hydroxyethyloxy-2-β-hydroxyethylamino-5-nitro-benzene
1-methoxy-2-β-hydroxyethylamino-5-nitrobenzene
1-β-hydroxyethyloxy-3-methylamino-4-nitrobenzene
1-β-dihydroxypropyloxy-3-methylamino-4-nitrobenzene
1-β-hydroxyethylamino-4-β,γ-dihydroxypropyloxy-2-nitrobenzene
1-β,γ-dihydroxypropylamino-4-trifluoromethyl-2-nitrobenzene
1-β-hydroxyethylamino-3-methyl-2-nitrobenzene
1-β-aminoethylamino-5-methoxy-2-nitrobenzene
1-hydroxy-2-chloro-6-amino-4-nitrobenzene
1-hydroxy-6-[bis(β-hydroxyethyl)amino]-3-nitrobenzene
1-β-hydroxyethylamino-2-nitrobenzene
1-hydroxy-4-β-hydroxyethylamino-3-nitrobenzene.

Among the azo direct dyes, mention may be made of the cationic azo dyes described in patent applications WO 95/15144, WO 95/01772 and EP 714 954, and FR 2 822 696, FR 2 825 702, FR 2 825 625, FR 2 822 698, FR 2 822 693, FR 2 822 694, FR 2 829 926, FR 2 807 650, WO 02/078 660, WO 02/100 834, WO 02/100 369 and FR 2 844 269, the content of which forms an integral part of the invention.

Among these compounds, mention may be made most particularly of the following dyes:
1,3-dimethyl-2-[[4-(dimethylamino)phenyl]azo]-1H-imidazolium chloride,
1,3-dimethyl-2-[(4-aminophenyl)azo]-1H-imidazolium chloride,
1-methyl-4-[(methylphenylhydrazono)methyl]pyridinium methyl sulfate.

Among the azo direct dyes that may also be mentioned are the following dyes described in the Colour Index International 3rd edition:

### Acid Yellow 9, Acid Black 1, Basic Red 22, Basic Red 76, Basic Yellow 57, Basic Brown 16, Acid Yellow 36, Acid Orange 7, Acid Red 33, Acid Red 35, Basic Brown 17, Acid Yellow 23, Acid Orange 24.

Mention may also be made of 4-hydroxy-3-(2-methoxy-phenylazo)-1-naphthalenesulfonic acid.

Among the quinone direct dyes that may be mentioned are the following dyes:

Acid Blue 62, Basic Blue 22, Basic Blue 99, and also the following compounds:
1-N-methylmorpholiniumpropylamino-4-hydroxyanthra-quinone
1,4-bis(β,γ-dihydroxypropylamino)anthraquinone.

Among the azine dyes that may be mentioned are the following compounds: Basic Blue 17, Basic Red 2.

Among the triarylmethane dyes, mention may be made of the following compounds:

### Basic Green 1, Acid Blue 9, Basic Violet 3, Basic Violet 14, Basic Blue 7, Acid Violet 49, Basic Blue 26, Acid Blue 7.

Among the natural direct dyes, mention may also be made of natural direct dyes such as lawsone, juglone, carminic acid, kermesic acid, purpurogallin, protocatechaldehyde, spinulosin and apigenidin. Extracts or decoctions containing these natural dyes may also be used, and especially henna-based poultices or extracts.

When this composition comprises direct dyes other than those with a logP of greater than 2, the composition may contain up to 20% of direct dyes. According to this particular embodiment, the composition of the invention comprises a total amount of direct dyes of between 0.001% and 15% by weight approximately.

The composition of the present invention may also contain oxidation bases and couplers conventionally used for oxidation dyeing.

Examples that may be mentioned include para-phenylenediamines, bis(phenyl)alkylenediamines, para-aminophenols, ortho-aminophenols and heterocyclic bases, and the addition salts thereof.

The couplers are, for example, meta-phenylenediamine couplers, meta-aminophenol couplers, meta-diphenol couplers, naphthalene-based couplers and heterocyclic couplers, and the addition salts thereof.

When they are present, the bases and couplers are each generally present in an amount of between 0.001% and 10% and preferably between 0.005% and 6% by weight approximately relative to the total weight of the dye composition.

The dye composition in accordance with the invention may also contain various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrating agents, sequestering agents, fragrances, buffers, dispersants, conditioners, for instance volatile or non-volatile, modified or unmodified silicones, film-forming agents, ceramides, preserving agents and opacifiers.

These above adjuvants are generally present in an amount for each of between 0.01% and 20% by weight relative to the weight of the composition.

For the dyeing of human keratin fibres, the dyeing medium is a cosmetic medium.

Needless to say, a person skilled in the art will take care to select this or these optional additional compound(s) such that the advantageous properties intrinsically associated with the oxidation dye composition in accordance with the invention are not, or are not substantially, adversely affected by the envisaged addition(s).

The pH of the dye composition in accordance with the invention is generally between 2 and 12 approximately, and preferably less than 7.

It may be adjusted to the desired value by means of acidifying or basifying agents usually used for dyeing keratin fibres, or alternatively using standard buffer systems.

Among the acidifying agents that may be mentioned, for example, are mineral or organic acids, for instance hydrochloric acid, orthophosphoric acid, sulfuric acid, carboxylic acids, for instance acetic acid, tartaric acid, citric acid or lactic acid, and sulfonic acids.

Among the basifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds of formula (III) below:

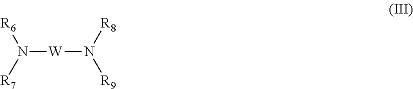

in which W is a propylene residue optionally substituted with a hydroxyl group or a C₁-C₄ alkyl radical; R₆, R₇, R₈ and R₉, which may be identical or different, represent a hydrogen atom or a C₁-C₄ alkyl or C₁-C₄ hydroxyalkyl radical.

A subject of the invention is also a dyeing process that consists in applying to keratin fibres a dye composition as defined above for a time that is sufficient to obtain the desired coloration. The leave-in time is generally between 1 and 60 minutes approximately and preferably 10 to 60 minutes approximately. After the leave-in time, the keratin fibres are rinsed, to reveal dyed fibres.

The composition of the invention may also comprise an oxidizing agent.

When the composition of the invention contains only direct dyes, this oxidizing agent makes it possible to obtain a lightening dyeing, i.e. simultaneous bleaching and dyeing of the hair.

When the dye composition comprises an oxidation base and/or a coupler, it is necessary to place this composition in contact with an oxidizing agent in order to form the coloured species. The oxidizing agents conventionally used for the oxidation dyeing of keratin fibres are, for example, hydrogen peroxide, urea peroxide, alkali metal bromates, persalts such as perborates and persulfates, peracids, and oxidase enzymes, among which mention may be made of peroxidases, 2-electron oxidoreductases such as uricases, and 4-electron oxygenases, for instance laccases. Hydrogen peroxide is particularly preferred.

The oxidizing agent may be added to the composition of the invention just at the time of use, or it may be used starting with an oxidizing composition containing it, which is applied simultaneously with or sequentially to the composition of the invention. The oxidizing composition may also contain various adjuvants conventionally used in hair dye compositions and as defined above.

The pH of the oxidizing composition containing the oxidizing agent is such that, after mixing with the dye composition, the pH of the resulting composition applied to the keratin fibres preferably ranges between 3 and 12 approximately and even more preferably between 5 and 11. It may be adjusted to the desired value by means of acidifying or basifying agents usually used in the dyeing of keratin fibres and as defined above.

The composition that is finally applied to the keratin fibres may be in various forms, such as in the form of liquids, creams or gels, or in any other form that is suitable for dyeing keratin fibres, and especially human hair.

The dyeing process may be performed at room temperature or at higher temperatures, for example by using a hairdryer, a drying hood, a smoothing iron, etc. According to one particular embodiment, the temperature is between room temperature and 200° C. and preferably between room temperature and 60° C.

The examples that follow serve to illustrate the invention without, however, being limiting in nature.

### EXAMPLES

### Example 1

The following composition was prepared:

| | |
|---|---|
| Propylene glycol n-propyl ether | 20% |
| Benzoic acid | 0.2% |
| Disperse Red 13 | 0.2% |
| Water | qs 100 |

The composition is applied to a lock of natural hair containing 90% white hairs, and also to a lock of permanent-waved hair containing 90% white hairs. After 30 minutes at room temperature, the locks are rinsed and dried. The hair is then dyed red.

### Example 2

The following composition was prepared:

| | |
|---|---|
| Ethanol | 10% |
| Tripropylene glycol methyl ether | 10% |
| Benzoic acid | 2% |
| Disperse Red 13 | 0.3% |
| Water | qs 100 |

The composition is applied to a lock of natural hair containing 90% white hairs, and also to a lock of permanent-waved hair containing 90% white hairs. After 30 minutes at room temperature, the locks are rinsed and dried. The hair is then dyed red.

1-21. (canceled)

22. A cosmetic dye composition comprising, in a cosmetically acceptable dyeing medium, at least one hydrophobic direct dye with a logP of greater than 2, and at least one propylene glycol derivative of formula (I):
R₁(OC₃H₆)ₙOR₂ (I)
wherein R₁ is chosen from a hydrogen atom, C₁-C₆ alkyl radicals, and C₂-C₆ acyl radicals; R₂ is chosen from C₁-C₆ alkyl radicals and C₆-C₃₀ aryl radicals; n is chosen from an integer ranging from 1 to 6; and the OC₃H₆ unit is chosen from linear OC₃H₆ and branched OC₃H₆ units; and further wherein the cosmetically acceptable dyeing medium comprises at least 40% by weight of water, relative to the total weight of the cosmetic dye composition.

23. The cosmetic dye composition according to claim 22, wherein the at least one hydrophobic direct dye with a logP of greater than 2 has a logP of greater than 4.

24. The cosmetic dye composition according to claim 22, wherein the at least one hydrophobic direct dye with a logP of greater than 2 is chosen from:

| Dye | Chemical structure | logP |
|---|---|---|
| Disperse Red 13 | 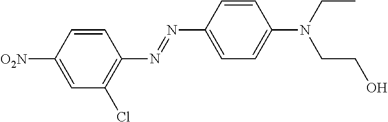 | 5.22 |
| Disperse Green 9 | 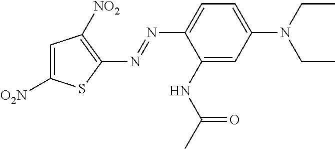 | 4.23 |
| Solvent Black 3 | 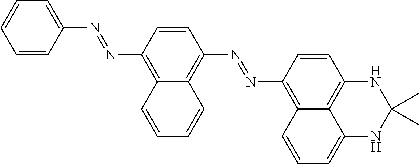 | 7.50 |
| Disperse Blue 148 | 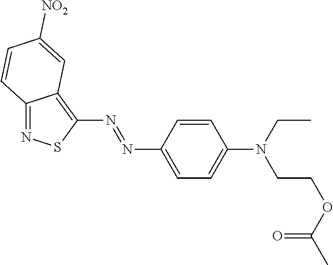 | 4.81 |
| Disperse Violet 63 | 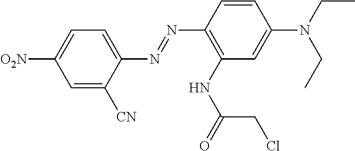 | 5.30 |
| Disperse Blue 60 | 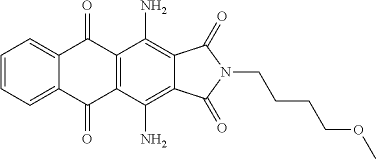 | 3.38 |
| Disperse Blue 14 | 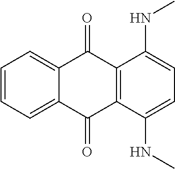 | 4.25 |
| Solvent Orange 15 | 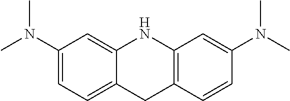 | 3.90 |
| Solvent Orange 7 | 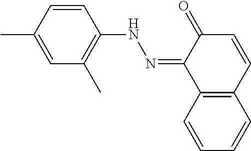 | 4.40 |
| Solvent Blue 14 | 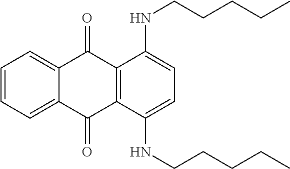 | 8.18 |
| Disperse Yellow 82 | 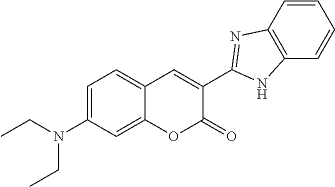 | 3.68 |

25. The cosmetic dye composition according to claim 22, wherein the at least one hydrophobic direct dye with a logP of greater than 2 is present in an amount ranging from 0.001% to 10% by weight relative to the total weight of the cosmetic dye composition.

26. The cosmetic dye composition according to claim 22, wherein n is chosen from an integer ranging from 1 to 4, and R₁ is chosen from C₁-C₆ alkyl radicals.

27. The cosmetic dye composition according to claim 26, wherein R₁ is chosen from C₁-C₄ alkyl radicals.

28. The cosmetic dye composition according to claim 22, wherein R₁ is a hydrogen atom, and n is chosen from an integer ranging from 2 to 6.

29. The cosmetic dye composition according to claim 22, wherein n is equal to 1, and R₂ is chosen from C₂-C₆ alkyl radicals.

30. The cosmetic dye composition according to claim 22, wherein the at least one propylene glycol derivative of formula (I) is present in an amount ranging from 0.1% to 40% by weight, relative to the total weight of the cosmetic dye composition.

31. The cosmetic dye composition according to claim 30, wherein the at least one propylene glycol derivative of formula (I) is present in an amount ranging from 1% to 20% by weight, relative to the total weight of the cosmetic dye composition.

32. The cosmetic dye composition according to claim 22, wherein the at least one propylene glycol derivative of formula (I) is chosen from:

| | |
|---|---|
| Dipropylene glycol methyl ether | CH₃O(C₃H₆O)₂H |
| Tripropylene glycol methyl ether | CH₃O(C₃H₆O)₃H |
| Propylene glycol methyl ether acetate | CH₃OC₃H₆OCOCH₃ |
| Dipropylene glycol methyl ether acetate | CH₃O(C₃H₆O)₂COCH₃ |
| Propylene glycol n-propyl ether | C₃H₇OCH₂CH(CH₃)OH |
| Propylene glycol n-butyl ether | C₄H₉OCH₂CH(CH₃)OH |
| Propylene glycol phenyl ether | C₆H₅OC₃H₆OH |

33. The cosmetic dye composition according to claim 22, wherein the cosmetically acceptable dye medium comprises at least 70% by weight of water, relative to the total weight of the cosmetic dye composition.

34. The cosmetic dye composition according to claim 22, further comprising at least one additional direct dye chosen from neutral, acidic or cationic nitrobenzene direct dyes; neutral, acidic or cationic azo direct dyes; neutral, acidic or cationic quinone direct dyes; azine direct dyes; triarylmethane direct dyes; indoamine direct dyes; and natural direct dyes.

35. The cosmetic dye composition according to claim 34, wherein the at least one direct dye is chosen from anthraquinone direct dyes.

36. The cosmetic dye composition according to claim 22, further comprising at least one oxidation base chosen from para-phenylenediamines, bis(phenyl)alkylenediamines, para-aminophenols, ortho-aminophenols, heterocyclic bases, and the addition salts thereof.

37. The cosmetic composition according to claim 36, wherein the at least one oxidation base is present, for each oxidation base, in an amount ranging from 0.001% to 10% by weight relative to the total weight of the cosmetic dye composition.

38. The cosmetic dye composition according claim 22, further comprising at least one coupler chosen from meta-phenylenediamines, meta-aminophenols, meta-diphenols, naphthalene-based couplers, heterocyclic couplers, and the addition salts thereof.

39. The cosmetic dye composition according to claim 38, wherein the at least one coupler is present in an amount, for each coupler, ranging from 0.001% to 10% by weight relative to the total weight of the cosmetic dye composition.

40. The cosmetic dye composition according to claim 22, comprising at least one adjuvant chosen from anionic, cationic, nonionic, amphoteric and zwitterionic surfactants; anionic, cationic, nonionic, amphoteric and zwitterionic polymers; mineral and organic thickeners; anionic, cationic, nonionic and amphoteric polymeric associative thickeners; antioxidants; penetrants; sequestrants; fragrances; buffers; dispersants; conditioning agents; film-forming agents; ceramides; preserving agents; and opacifiers.

41. The cosmetic dye composition according to claim 40, wherein the at least one adjuvant is present in an amount, for each adjuvant, ranging from 0.01% to 20% by weight relative to the weight of the cosmetic dye composition.

42. The cosmetic dye composition according to claim 22, further comprising at least one oxidizing agent.

43. A process for dyeing keratin fibers, comprising:
applying to the keratin fibers a cosmetic dye composition,
wherein the cosmetic dye composition comprises, in a cosmetically acceptable dyeing medium, at least one hydrophobic direct dye with a logP of greater than 2, and at least one propylene glycol derivative of formula (I):
R₁(OC₃H₆)ₙOR₂ (I)
wherein R₁ is chosen from a hydrogen atom, C₁-C₆ alkyl radicals, and C₂-C₆ acyl radicals; R₂ is chosen from C₁-C₆ alkyl radicals and C₆-C₃₀ aryl radicals; n is chosen from an integer ranging from 1 to 6; and the OC₃H₆ unit is chosen from linear OC₃H₆ and branched OC₃H₆ units; and further wherein the cosmetically acceptable dyeing medium comprises at least 40% by weight of water, relative to the total weight of the cosmetic dye composition;
leaving the cosmetic dye composition on the keratin fibers for a period of time that is sufficient to obtain the desired coloration, and
rinsing the keratin fibers.

44. A process for preparing a cosmetic dye composition, comprising, combining
a cosmetically acceptable dyeing medium comprising at least 40% by weight of water, relative to the total weight of the cosmetic dye composition, and
at least one hydrophobic direct dye with a logP of greater than 2, and
at least one propylene glycol derivative of formula (I):
R₁(OC₃H₆)ₙOR₂ (I)
wherein R₁ is chosen from a hydrogen atom, C₁-C₆ alkyl radicals, and C₂-C₆ acyl radicals; R₂ is chosen from C₁-C₆ alkyl radicals and C₆-C₃₀ aryl radicals; n is chosen from an integer ranging from 1 to 6; and the OC₃H₆ unit is chosen from linear OC₃H₆ and branched OC₃H₆ units.

45. A multi-compartment kit for dyeing keratin fibers, comprising at least two compartments, wherein the at least one first compartment comprises a cosmetic dye composition, comprising, in a cosmetically acceptable dyeing medium, at least one hydrophobic direct dye with a logP of greater than 2, and at least one propylene glycol derivative of formula (I):
R₁(OC₃H₆)ₙOR₂ (I)
wherein R₁ is chosen from a hydrogen atom, C₁-C₆ alkyl radicals, and C₂-C₆ acyl radicals; R₂ is chosen from C₁-C₆ alkyl radicals and C₆-C₃₀ aryl radicals; n is chosen from an integer ranging from 1 to 6; and the OC₃H₆ unit is chosen from linear OC₃H₆ and branched OC₃H₆ units; and further wherein the cosmetically acceptable dyeing medium comprises at least 40% by weight of water, relative to the total weight of the cosmetic dye composition; and
the at least one second compartment comprises a composition comprising an oxidizing agent.
